(19) United States
(12) Patent Application Publication
(10) Pub. No.: US 2024/0415400 A1
(43) Pub. Date: Dec. 19, 2024

(54) METHOD AND SYSTEM FOR PEDIATRIC HEARTBEAT MONITORING

(71) Applicant: VITASCOPE INC., Toronto (CA)

(72) Inventors: Sridhar Krishnan, Richmond Hill (CA); Abdelrahman Abdou, Oakville (CA); Niraj Mistry, Toronto (CA); Douglas M. Campbell, Whitby (CA)

(21) Appl. No.: 18/705,259

(22) PCT Filed: Oct. 27, 2022
(86) PCT No.: PCT/CA2022/051597

§ 371 Date: Apr. 26, 2024

Related U.S. Application Data
(60) Provisional application No. 63/272,902, filed on Oct. 28, 2021.

(51) Int. Cl. A61B 5/0245; A61B 5/00; A61B 5/352

(52) U.S. Cl. CPC A61B 5/0245; A61B 5/352; A61B 5/6823; A61B 5/7203; A61B 2503/045; A61B 2562/125

(57) ABSTRACT

Various embodiments provided herein relate to a method and system for pediatric heartbeat monitoring. In at least one embodiment, the system comprises at least one electrode configured to be applied to a pediatric subject; at least one processor coupled to the at least one electrode, the at least one processor configured for: receiving, from the at least one electrode, an input electrocardiogram (ECG) signal; determining a signal quality index (SQI) associated with input ECG signal; applying a bandpass filter to the input ECG signal to generate a filtered ECG signal; determining a derivative of the filtered ECG signal to generate a derived ECG signal; applying a squaring function to the derived ECG signal to generate a squared ECG signal; applying an integrator to the derived ECG signal to generate an integrated ECG signal; and applying one or more decision rules to the integrated ECG signal to output one or more heartbeat parameters associated with the subject.

### TECHNICAL FIELD

Various embodiments are described herein that relate to heartbeat monitoring, and in particular, to a method and system for pediatric heartbeat monitoring.

### BACKGROUND

The following is not an admission that anything discussed below is part of the prior art or part of the common general knowledge of a person skilled in the art.

Newborns and infants often require real-time or near real-time vital sign monitoring to detect critical medical conditions necessitating immediate medical intervention. To this end, an important vital to monitor are heart vitals. In many cases, an abnormal heartrate can signal imminent need for respiratory assistance or resuscitation. Current methods for neonatal heartbeat monitoring include, for example, using standard medical listening devices (i.e., stethoscopes), analyzing measurements from oxygen meters and/or probes, as well as using electrocardiogram (ECG) machines. There are, however, significant drawbacks such as lack of accuracy, patient comfort, robustness and speed associated with each of these conventional techniques.

### SUMMARY OF VARIOUS EMBODIMENTS

The following introduction is provided to introduce the reader to the more detailed discussion to follow. The introduction is not intended to limit to define any claim or as yet unclaimed invention. One or more inventions may reside in any combination or sub-combination of elements or process steps disclosed in any part of this document including its claims and figures.

In accordance with a broad aspect of the teachings herein, there is provided a system for pediatric heartbeat monitoring, comprising: at least one electrode configured to be applied to a pediatric subject; at least one processor coupled to the at least one electrode, the at least one processor configured for: receiving, from the at least one electrode, an input electrocardiogram (ECG) signal; determining a signal quality index (SQI) associated with input ECG signal; applying a bandpass filter to the input ECG signal to generate a filtered ECG signal; determining a derivative of the filtered ECG signal to generate a derived ECG signal; applying a squaring function to the derived ECG signal to generate a squared ECG signal; applying an integrator to the derived ECG signal to generate an integrated ECG signal; and applying one or more decision rules to the integrated ECG signal to output one or more heartbeat parameters associated with the subject.

In some embodiments, the at least one processor is configured to output the one or more heartbeat parameters in real-time, or near real-time.

In some embodiments, the system further comprises a hardware circuit filter coupled between the at least one electrode and the at least one processor, wherein the hardware circuit filter receives an ECG signal from the at least one electrode and generates a pre-filtered ECG signal, and the input ECG signal comprises the pre-filtered ECG signal.

In some embodiments, the hardware circuit filter is a bandpass filter.

In some embodiments, the bandpass filter has a passband range of 3 to 48 Hz.

In some embodiments, the bandpass filter has a passband range of 5 to 15 Hz.

In some embodiments, the one or more output heartbeat parameters comprise one or more of heartrate, heartrate variability, RR interval and R-peak locations, and an indication of abnormal heartrate.

In some embodiments, the SQI is one of kurtosis SQI (kSQI), skewness SQI (sSQI), a histogram, an activity measure, a mobility measure, a signal to noise ratio (SNR), a LZW complexity measure and a fractal dimension measure.

In some embodiments, the input ECG signal is a 12-bit ECG signal.

In some embodiments, the at least one processor is further configured to apply thresholding to the integrated ECG signal and to further convert the signal into a one-bit signal.

In some embodiments, the at least one processor is further configured to: encode the one or more output heartbeat parameters using a varied Lempel-Ziv encoding algorithm to generate encoded output parameters; and transmit the one or more encoded output parameters to an external device.

In some embodiments, the at least one electrode comprises a 3D printed dry electrodes printed from conductive polylactic acid (PLA) film.

In some embodiments, each of the at least one 3D printed dry electrodes has length, height and width dimensions of 32 millimeters, 18 millimeters and 6 millimeters, respectively.

In some embodiments, the dry electrodes are manufactured using a nozzle temperature of about 215° C., a heated bed temperature of about 60° C., a print speed of about 25 mm/s and a fill ratio of 100%.

In accordance with another broad aspect of the teachings herein, there is provided a method for pediatric heartbeat monitoring comprising: receiving, from the at least one electrode, an input electrocardiogram (ECG) signal; determining a signal quality index (SQI) associated with input ECG signal; applying a bandpass filter to the input ECG signal to generate a filtered ECG signal; determining a derivative of the filtered ECG signal to generate a derived ECG signal; applying a squaring function to the derived ECG signal to generate a squared ECG signal; applying an integrator to the derived ECG signal to generate an integrated ECG signal; and applying one or more decision rules to the integrated ECG signal to output one or more heartbeat parameters associated with the subject.

In some embodiments, the one or more heartbeat parameters are output in real-time, or near real-time.

In some embodiments, the method further comprises receiving an ECG signal from the at least one electrode and generating, via a hardware circuit filter, a pre-filtered ECG signal, and the input ECG signal comprises the pre-filtered ECG signal.

In some embodiments, the hardware circuit filter is a bandpass filter.

In some embodiments, the bandpass filter has a passband range of 3 to 48 Hz.

In some embodiments, the bandpass filter has a passband range of 5 to 15 Hz.

In some embodiments, the one or more output heartbeat parameters comprise one or more of heartrate, heartrate variability, RR interval and R-peak locations, and an indication of abnormal heartrate.

In some embodiments, the SQI is one of kurtosis SQI (kSQI), skewness SQI (sSQI), a histogram, an activity measure, a mobility measure, a signal to noise ratio (SNR), a LZW complexity measure and a fractal dimension measure.

In some embodiments, the input ECG signal is a 12-bit ECG signal.

In some embodiments, the method further comprises applying thresholding to the integrated ECG signal and converting the signal into a one-bit signal.

In some embodiments, the method further comprises encoding the one or more output heartbeat parameters using a varied Lempel-Ziv encoding algorithm to generate encoded output parameters; and transmitting the one or more encoded output parameters to an external device.

In some embodiments, the at least one electrode comprises a 3D printed dry electrodes printed from conductive polylactic acid (PLA) film.

In some embodiments, each of the at least one 3D printed dry electrodes has length, height and width dimensions of 32 millimeters, 18 millimeters and 6 millimeters, respectively.

In some embodiments, the dry electrodes are manufactured using a nozzle temperature of about 215° C., a heated bed temperature of about 60° C., a print speed of about 25 mm/s and a fill ratio of 100%.

Other features and advantages of the present application will become apparent from the following detailed description taken together with the accompanying drawings. It should be understood, however, that the detailed description of the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 is an example embodiment of a system for pediatric heartbeat monitoring;

FIG. 2 is a simplified block diagram for an example embodiment of a hardware architecture for a monitoring device and a computer terminal or external server;

FIG. 3 is a process flow for an example embodiment of a method for pediatric heartbeat monitoring;

FIG. 4 is an example embodiment of a process flow for a method for analyzing electrocardiogram (ECG) data to determine one or more heartbeat parameters;

FIG. 5 is a process flow for an example embodiment for a method for signal compression;

FIG. 6A is a representation of a 12-bit ECG signal, according to an example embodiment;

FIG. 6B is a representation of a 1-bit ECG signal, according to an example embodiment;

FIG. 7 illustrates different perspective and elevation views of an example embodiment of a three-dimensional (3D) printed dry electrode;

FIG. 8A shows an example plot of ECG signals acquired from a single 3D printed dry electrode applied to a pediatric subject for a thirty second acquisition interval;

FIG. 8B shows an example plot of ECG signals acquired from a single 3D printed dry electrode applied to a pediatric subject for a five minute acquisition interval;

FIG. 9 is an example plot of a snapshot of two ECG cycles and labelled with the QRS complex and T-wave;

FIG. 10A shows an example histogram plot generated from the plot in FIG. 8A; and

FIG. 10B shows an example histogram plot generated from the plot in FIG. 8B.

Further aspects and features of the example embodiments described herein will appear from the following description taken together with the accompanying drawings.

### DETAILED DESCRIPTION OF THE EMBODIMENTS

Various embodiments in accordance with the teachings herein will be described below to provide an example of at least one embodiment of the claimed subject matter. No embodiment described herein limits any claimed subject matter. The claimed subject matter is not limited to devices, systems or methods having all of the features of any one of the devices, systems or methods described below or to features common to multiple or all of the devices, systems or methods described herein. It is possible that there may be a device, system or method described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

It will be appreciated that for simplicity and clarity of illustration, where considered appropriate, reference numerals may be repeated among the figures to indicate corresponding or analogous elements or steps. In addition, numerous specific details are set forth in order to provide a thorough understanding of the example embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the example embodiments described herein.

It should also be noted that the terms “coupled” or “coupling” as used herein can have several different meanings depending in the context in which these terms are used. For example, the terms coupled or coupling can have a mechanical, fluidic or electrical connotation. For example, as used herein, the terms coupled or coupling can indicate that two elements or devices can be directly connected to one another or connected to one another through one or more intermediate elements or devices via an electrical or magnetic signal, electrical connection, an electrical element or a mechanical element depending on the particular context. Furthermore, coupled electrical elements may send and/or receive data.

Unless the context requires otherwise, throughout the specification and claims which follow, the word “comprise” and variations thereof, such as, “comprises” and “comprising” are to be construed in an open, inclusive sense, that is, as “including, but not limited to”.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5% or 10%, for example, if this deviation does not negate the meaning of the term it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as 1%, 2%, 5%, or 10%, for example.

Reference throughout this specification to “one embodiment”, “an embodiment”, “at least one embodiment” or “some embodiments” means that one or more particular features, structures, or characteristics may be combined in any suitable manner in one or more embodiments, unless otherwise specified to be not combinable or to be alternative options.

As used in this specification and the appended claims, the singular forms “a,” “an,” and “the” include plural referents unless the content clearly dictates otherwise. It should also be noted that the term “or” is generally employed in its broadest sense, that is, as meaning “and/or” unless the content clearly dictates otherwise.

The headings and Abstract of the Disclosure provided herein are for convenience only and do not interpret the scope or meaning of the embodiments.

Similarly, throughout this specification and the appended claims the term “communicative” as in “communicative pathway,” “communicative coupling,” and in variants such as “communicatively coupled,” is generally used to refer to any engineered arrangement for transferring and/or exchanging information. Examples of communicative pathways include, but are not limited to, electrically conductive pathways (e.g., electrically conductive wires, electrically conductive traces), magnetic pathways (e.g., magnetic media), optical pathways (e.g., optical fiber), electromagnetically radiative pathways (e.g., radio waves), or any combination thereof. Examples of communicative couplings include, but are not limited to, electrical couplings, magnetic couplings, optical couplings, radio couplings, or any combination thereof.

Throughout this specification and the appended claims, infinitive verb forms are often used. Examples include, without limitation: “to detect,” “to provide,” “to transmit,” “to communicate,” “to process,” “to route,” and the like. Unless the specific context requires otherwise, such infinitive verb forms are used in an open, inclusive sense, that is as “to, at least, detect,” to, at least, provide,” “to, at least, transmit,” and so on.

In addition, it should be noted that the example embodiments of the systems and methods described herein may be implemented as a combination of hardware or software. In some cases, the example embodiments described herein may be implemented, at least in part, by using one or more computer programs, executing on one or more programmable devices comprising at least one processing element, and a data storage element (including volatile memory, non-volatile memory, storage elements, or any combination thereof). These devices may also have at least one input device (e.g. a keyboard, mouse, touchscreen, or the like), and at least one output device (e.g. a display screen, a printer, a wireless radio, or the like) depending on the nature of the device.

As explained in the background, heartbeat monitoring for neonatal or pediatric applications is important in detecting critical medical conditions requiring immediate medical intervention, i.e., respiratory assistance or resuscitation, typically in less than a minute after birth. However, existing methods for pediatric heartbeat monitoring suffer from a number of important drawbacks.

One conventional method for monitoring pediatric heartbeat involves the use of medical listening devices (i.e., a stethoscopes). Auditory monitoring, however, presents challenges in high ambient noise settings, e.g., hospital rooms. There is also an inherent subjectivity in determining whether an infant's heartrate is abnormal based only on auditory observation. Another method for monitoring pediatric heartbeat involves the use of oxygen meters and/or probes, such as pulse oximeters. Oxygen probes monitor heartrate based on detected oxygen levels. Pulse oximeters use reflected infrared and/or other sources of light to monitor heartrate. In many cases, however, oxygen probes require at least a few minutes to generate results, which is not ideal for medical conditions requiring time critical invention.

Still yet another method for neonatal heartrate monitoring involves the use of electrocardiogram (ECG) machines. While ECG machines are useful for accurate and rapid monitoring, ECG machines are also prohibitively expensive, and may not always be readily available in all hospital rooms. Also, it requires a long time, more than 1 minute, to set electrodes on the skin and start collecting ECG and calculating heartrate. To this end, conventional ECG machines use “wet” ECG probes, which require applying fluid to a subject's skin. In pediatric application, fluid application may be difficult (i.e., the fluid may not “stick” to the skin), and may otherwise bruise a newborn's sensitive skin.

In view of the foregoing, embodiments herein provide for a method and system for neonatal heartbeat monitoring that mitigate at least some of the aforementioned drawbacks inherent in conventional monitoring systems.

In accordance with at least one embodiment, the disclosed methods and systems provide for portable and cost-effective point-of-care heartbeat monitoring for newborns and infants. As provided herein, the methods and systems can determine neonatal heartbeat parameters in real-time or near real-time, and with minimal latency and delay. This, in turn, facilitates immediate intervention when critical conditions are detected.

In accordance with other embodiments provided herein, the disclosed methods and systems further allow monitoring of neonatal heartbeat using three-dimensional (3D) printed dry ECG electrodes. The dry ECG electrodes are characterized by small form factor and address drawbacks associated with the use of wet ECG electrodes, especially with pediatric subjects. In at least some embodiments, the dry 3D ECG electrodes are manufactured using readily available and cost-effective material suitable for mass or volume production and without significant compromises to the quality of the captured ECG signal.

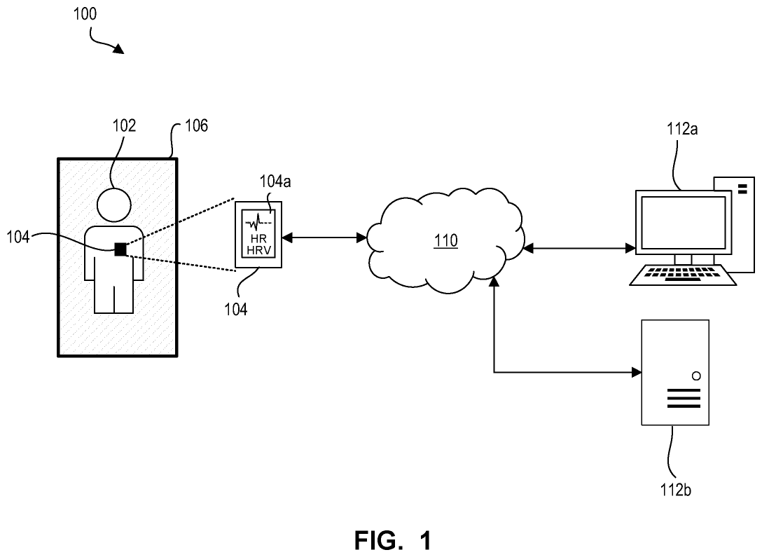

Reference is now made to FIG. 1, which shows a simplified block diagram of an example embodiment of a system 100 for pediatric heartbeat monitoring.

As shown, a pediatric subject 102 (e.g., a newborn or infant positioned on a bed or horizontal surface 106) is monitored using a heartbeat monitoring device 104. Monitoring device 104 may provide point-of-care monitoring of the subject's heart vitals, including generating estimates of various heartbeat parameters. In at least one embodiment, the device 104 is a portable device which can be deployed by medical practitioners in medical settings, or otherwise by consumers in more casual settings.

In the illustrated example, the monitoring device 104 is positioned around the subject's chest area. The monitoring device 104 may include one or more electrodes to collect ECG data (i.e., ECG probes). For example, the monitoring device 104 may include only a single-lead electrode. In other cases, any other number of electrodes can be provided in the monitoring device 104. In at least one embodiment, the electrode(s) in the monitoring device 104 are novel small form factor 3D printed dry electrodes. As previously explained, dry electrodes may at least partially mitigate drawbacks associated with the use of conventional wet electrodes in neonatal applications. As further provided herein, the dry electrodes may be configured to generate high quality ECG with minimal noise interference.

In operation, monitoring device 104 receives ECG signals acquired by ECG electrodes, and processes the signals locally, or in-situ, to determine one or more subject-specific heartbeat monitoring parameters. The heartbeat parameters can include, for example, the subject's instantaneous heartrate (HR), heartrate variability (HRV), heartbeat peak locations, RR distances, as well as a general indication of whether heartrate is in an abnormal and/or normal range. In at least one embodiment, the monitoring device 104 may estimate the heartbeat parameters from ECG data using low-power artificial intelligence (AI) or machine learning models, presented on the device, through on-chip computing and/or on the cloud using cloud computing.

Monitoring device 104 can output the determined heartbeat parameters using various methods. In one example embodiment, parameters can be output on a display 104a of the monitoring device 104. Accordingly, a user can observe and monitor heartbeat parameters using the device display 104a. In other cases, the heartbeat parameters and/or raw signal data can be transmitted, over a network 110, to a remote computer terminal 112a. Computer terminal 112a can further process the received data and/or display raw or processed data on a corresponding display interface. In still other cases, the heartbeat parameters and/or raw signal data can be transmitted to an external server 112b (i.e., a cloud server), for further processing or storage.

In at least some embodiments, monitoring device 104 may output heartbeat monitoring parameters in real-time or near real-time. In turn, this may allow medical staff—or general device users—to also track heartrate abnormalities in subjects in real-time or near real-time. For example, by simply observing the monitoring device 104, a practitioner or casual consumer can be alerted of critical medical conditions requiring time-sensitive intervention.

To this end, the inventors have determined a number of challenges in realizing real-time, or near real-time, heartrate monitoring using a portable device. In particular, owing to their small form factor, portable devices typically have limited processing power and low memory capabilities. Accordingly, these devices incur significant delay and latency when executing complex algorithms, such as the algorithms required to process and analyze ECG data. Complex algorithms also deplete the portable device's small energy reserves (i.e., batteries), thereby making portable devices ill-suited for continuous monitoring for extended time durations. For these reasons, among others, real-time or near real-time point-of-care neonatal heartbeat monitoring is difficult to realize with portable devices. Larger desktop computers 112a and/or servers 112b are therefore relied on for their stronger processing capabilities.

In the context of using portable devices to analyze ECG data, the inventors have further appreciated that the complexity of processing ECG data stems from two primary factors: (i) the complex software modules typically required to de-noise (or filter) the input ECG signal; and (ii) the complex estimation models required to analyze the filtered ECG signal to generate high accuracy output estimates of heartbeat parameters.

In an effort to overcome at least some of the aforementioned challenges, and in accordance with embodiments provided herein, portable monitoring device 104 can incorporate one or more of the following features: (a) a hardware signal filter; and (b) a low power estimation model for estimating heartbeat parameters.

As explained in greater detail, the hardware signal filter provides initial de-noising of the input ECG signal. Once the hardware filter preprocesses the signal, the signal is digitized and transmitted to the monitoring device processor. As the processor is receiving a pre-filtered signal, the device processor is able to execute a less complex software filter to remove any residue noise artifacts. In other words, less processing power and energy is required to de-noise the already pre-filtered ECG signal. The inventors have therefore appreciated a novel use for hardware signal filters in lessening the computational burden on portable device processor in performing software-based signal filtering.

By a similar token, the inventors have further realized a low power, low complexity machine learning model that can estimate heartbeat parameters from filtered ECG signals. The low power, low complexity model enables high accuracy estimations while also reducing the computation burden on the portable device processor.

Accordingly, the combination of the hardware pre-filtering circuit and low-power estimation model is believed to address challenges inherent in using of portable devices for real-time, or near-real-time, pediatric heartbeat monitoring. That is, the combination of these features lessens the processing, memory and energy resources required to implement point-of-care pediatric heartbeat monitoring with low voltage processors commonly found in small portable devices.

Continuing with reference to FIG. 1, network 110 may be a wired or wireless network, and may connect to the internet. Typically, the connection between network 110 and the internet may be made via a firewall server (not shown). In some cases, there may be multiple links or firewalls, or both, between network 110 and the Internet. Some organizations may operate multiple networks 110 or virtual networks 110, which can be internetworked or isolated. These have been omitted for ease of illustration, however it will be understood that the teachings herein can be applied to such systems. Network 110 may be constructed from one or more computer network technologies, such as Bluetooth™, IEEE 802.3 (Ethernet), IEEE 802.11 and similar technologies. Network 110 may be developed in accordance with HL7, HIPAA in the United States, PIPEDA in Canada, GDPR in Europe and similar protocols.

Remote computer terminal 112a may be any desktop, portable, mobile or laptop computer that can receive raw and/or processed data from the monitoring device 104. For example, the computer terminal 112 can be associated with a third-party and can allow for further analysis or processing of the data.

Server 112b is a computer server that is connected to network 110. Server 112b has a processor, volatile and non-volatile memory, at least one network interface, and may have various other input/output devices. As with all devices shown in the environment 100, there may be multiple servers 110b, although not all are shown. It will be understood that the server 112b need not be a dedicated physical computer. For example, in various embodiments, the various logical components that are shown as being provided on server 112b may be hosted by a third party “cloud” hosting service such as Amazon™ Web Services™ Elastic Compute Cloud (Amazon EC2).

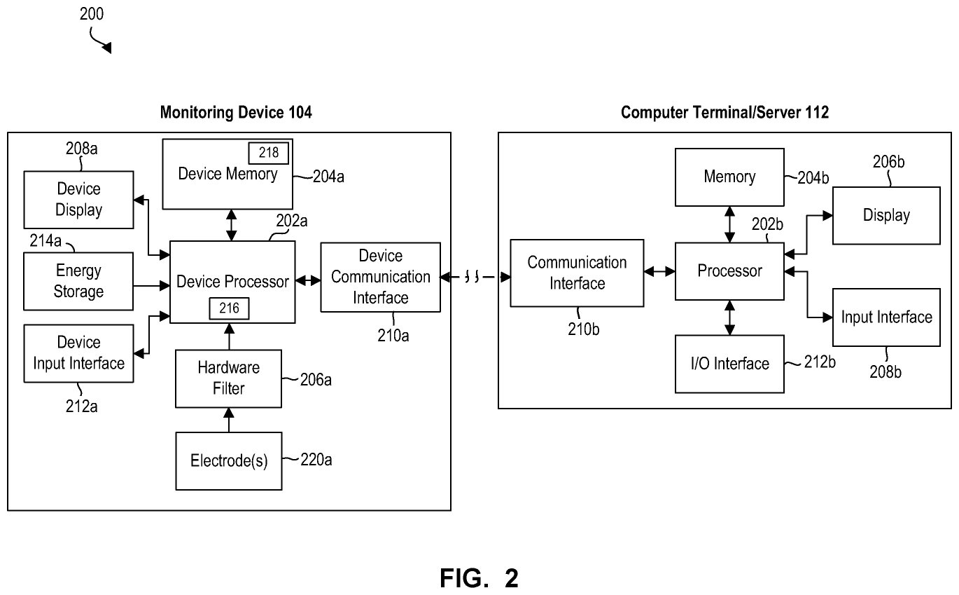

Reference is now made to FIG. 2, which illustrates simplified block diagrams for an example embodiment of the hardware architecture of the monitoring device 104 and the computer terminal 112a and/or server 112b.

As shown, the monitoring device 104 may include a device processor 202a which is coupled to one or more of a device memory 204a, filtering hardware 206a, a device display 208a, a communication interface 210a, a device input interface 212a and an energy storage unit 214a.

Processor 202a is a computer processor, such as a general purpose microprocessor. In some other cases, processor 202a may be a field programmable gate array, application specific integrated circuit, microcontroller, or other suitable computer processor. As used herein, processor 202a may comprise a single processor or may comprise multiple processors. In at least one embodiment, the processor 202a is an STM32WB55 microcontroller manufactured by STMicroelectronics™ and which supports Bluetooth® LE, Zigbee® and Thread® wireless connectivity. The STM32WB55 microcontroller may have an ultra-lower-power dual Arm Rotex-M4 MCU 64 Hz, Cortex-MO+32 MHz with Flash memory in a range of 254 KB to 1 Mbyte. In various cases, processor 202a may be in a class of low or ultra-low voltage processors (ULV) processors that are underclocked to consume low power (i.e., 17 Watts or below), as is understood in the art. In other cases, the processor 202a may be in a class of processors that consume low mAH current per number of clock cycles.

In some cases, processor 202a may comprise a microcontroller or such that includes functionality of an analog-to-digital (ADC) converter 216 to convert received analog signals to digital signals. In some embodiments, the ADC converter 216 may be operable to convert a received analog signal (e.g., an ECG signal) into a simplified 12-bit signal which is processable with low power computation processing algorithms. In other cases, the ADC converter hardware may be provided separately from the processor 202a.

Processor 202a is coupled, via a computer data bus, to memory 204a. Memory 204a may include both volatile and non-volatile memory. Non-volatile memory stores computer programs consisting of computer-executable instructions, which may be loaded into volatile memory for execution by processor 202a as needed. It will be understood by those of skill in the art that references herein to monitoring device 104 as carrying out a function or acting in a particular way imply that processor 202a is executing instructions (e.g., a software program) stored in memory 204a and possibly transmitting or receiving inputs and outputs via one or more interface. Memory 204a may also store data input to, or output from, processor 202a in the course of executing the computer-executable instructions.

In at least one embodiment, the memory 204a may store a signal analysis program 218. Signal analysis program 218 may include a “tiny” or “edge” AI model, which is applied to raw ECG signals to generate estimated heartbeat parameters. The “tiny” AI model is so-called for being a low-computation, low-complexity machine learning model adapted for execution by a low-power processor 202a of the portable device 104. This is in contrast to higher complexity estimation models that require high processing and memory resources. In at least some embodiments, the signal analysis program 218 may also perform software-based filtering of received ECG signals, as well as other functions provided herein.

Monitoring device 104 can also include a hardware circuit filter 206a. The hardware filter 206a may be connected to one or more electrode(s) 220a of the monitoring device. Hardware filter 206a can receive ECG signals from the electrode(s) 220a, and pre-filter the signals prior to digitizing and transmitting the signals to the device processor 202a. As noted previously, the filtering hardware 206a can reduce the complexity of software-based signal filtering by the signal analysis program 218 executing on the device processor 202a.

In at least some embodiments, the hardware filter 206a can be a bandpass filter (BPF). The BPF filter 206a may pass a select bandpass range of frequencies that can eliminate, for example, power line interference, movement artifacts, and high frequency (HF) noise prior to signal digitization. In at least one embodiment, the bandpass range may be between 3 to 48 Hz. The selection of this frequency range is in recognition that most noises and artifacts—as they relate to neonatal heartbeat monitoring—can be eliminated at this level. The BPF filter may be further configured to amplify the received signal by a pre-defined gain, i.e., a gain of 100× or 115×. In some cases, the gain may be adjusted based on the particular application, as well as the nature of the electrodes 220a. In at least one embodiment, the hardware filter may be implemented using an Analog Front-End (AFE) AD8232 chip manufactured by Analog Devices™, or otherwise using any other known hardware implementation such as other 1^{st} order or higher passive and/or active filters using capacitors, resistors, and other AFE chips. Examples of hardware filter architectures can include, by way of non-limiting examples, Butterworth, Chebyshev, Bessel, Elliptic, Gaussian, Linkwitz-Riley, and Optimum “L” (Legere) filters.

One or more electrode(s) 220a may also be included in the monitoring device 104. In at least one embodiment, the electrode(s) 220a are dry electrodes, which may be better suited for neonatal applications, as previously explained. In at least some further embodiments, the electrode(s) 220a may be 3D printed dry electrode(s). In other cases, the electrode(s) may not be integral with the monitoring device 104, but may be separate external components connected to the monitoring device 104.

Device display interface 208a may include any interface (i.e., LED screen, etc.) that can be used to display various information, i.e., heartrate monitoring parameters in real-time or near real-time.

Device communication interface 210a is one or more data network interface, such as an IEEE 802.3 or IEEE 802.11 interface, for communication over a network.

Device input interface 212a may be any hardware device that may receive user inputs, and may include buttons, etc. In some cases, the display interface 208a may also function as an input interface 212a as the case may be, for example, in a capacitive touchscreen display.

Energy storage unit 214a can be any component for storing energy (i.e., power) to power the monitoring device 104. In various cases, the energy storage unit 214a can be a small battery, such as a coin cell, normal AAA, Li-po, and/or any other low-power batteries. The capacity of the energy storage unit 214a can range from 500 mAh to 1500 mAh, and the battery can be a 1.5V to 3V battery.

Computer terminal 112a and/or server 112b may also include a processor 202b coupled to one or more of a memory 204b, a display 206b, an input interface 208b, a communication interface 210b and/or a input/output (I/O) interface 212b.

In at least one embodiment, the display 206b of the computer terminal 112a and/or server 112b may be used display graphical user interfaces (GUI) which can display raw and/or processed data received from the monitoring device 104.

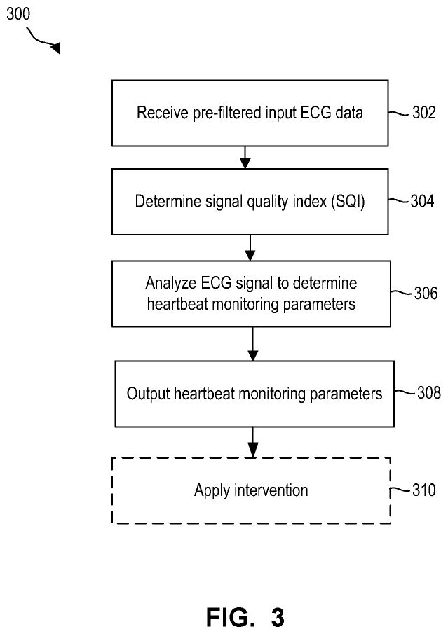

Reference is now made to FIG. 3, which shows a process flow for an example embodiment of a method 300 for pediatric heartbeat monitoring. Method 300 may be performed, for example, by device processor 202a of monitoring device 104. For example, method 300 may be performed as the device processor 202a is executing the signal analysis program 218.

At 302, input ECG signal data may be received from one or more electrodes 220a. For example, ECG signals may be received from electrode(s) 220a placed in contact with the pediatric subject's skin. Electrode(s) 220a may be positioned around the subject's chest area (i.e., towards a lower portion of the pediatric subject's sternum), as this position is closest to the heart, and can result in ECG signals with the highest amplitude. In some cases, a waiting period of 10 seconds may be required before obtaining ECG data so as to account for an ECG settling time.

In at least some embodiments, the input ECG data—received by the processor 202a—is pre-filtered by the hardware filter 206a. As stated previously, this can remove noise artifacts to reduce software-based filtering by the signal analysis program 218. The pre-filtered ECG signal may also be digitized by the ADC 216. In some cases, the ADC 216 can digitize the signal to generate a 12-bit digitized signal.

At 304, a signal quality index (SQI) can be determined in respect of the received ECG signal. In general, the SQI is a measure of the reliability and soundness of the obtained ECG signal. For example, in various cases, the SQI can indicate whether the signal is excessively noisy. As provided herein, the SQI can be a factor in determining whether the received ECG signal is clinically suitable for generating accurate estimates of a subject's heartbeat parameters.

Common SQIs that can be determined at act 304 can include various statistical measures computed on the signal including kurtosis SQI (kSQI), skewness SQI (sSQI), histograms, activity, mobility and signal to noise ratio (SNR).

More particularly, kurtosis SQI (kSQI) is used to determine the Gaussianity of a signal distribution. ECG signals are known to be hyper-Gaussian which exemplifies that higher kSQI values represent lower quality ECG. On the other hand, skewness SQI (sSQI) is defined as the examination of the symmetry behavior of a distribution. sSQI can be used in ECG to determine whether the signals are heavily tailed, values above −1 or 1, or moderately tailed values range between −0.5 and −1 or 0.5 and 1. sSQI with values below 0.5 or above −0.5 identify the distribution as approximately symmetrical. Tail behavior is associated with noise context in the signal where heavily tailed ECG signals show more noise than moderately tailed signals. Histograms assist in visualizing the distribution of the signal, and they are valuable in examining the skewness visually. Activity and mobility of the signal can be used to see the randomness of the signal where activity is defined as the variance of the signal and mobility the square root of ratio of the first derivative variance to original signal variance. As both values increase for an ECG, the higher the noise presence of different noise sources is detected. Lastly, SNR is defined as the measure of the desired signal power to the noise power of the signal. This ratio determines the overall quality of the ECG signal in comparison to its noise in the form of decibels (dB).

Various other SQI techniques can be determined at act 304, including determining fractal dimensions and/or Lempel-Ziv complexity of the ECG signal.

In some cases, the window for determining the SQI may be similar to the size of the signal buffer processed on the device 104. In some embodiments, the device 104 may have a window that can range from 2 seconds to 10 seconds to acquire an acceptable heartrate (HR) and SQI concurrently. In some cases, the device may use a very short term signal (i.e., a few R-R intervals) to get a good estimate. This may be compared to conventional ECG machine that require more time to obtain a good estimate of heartbeat parameters.

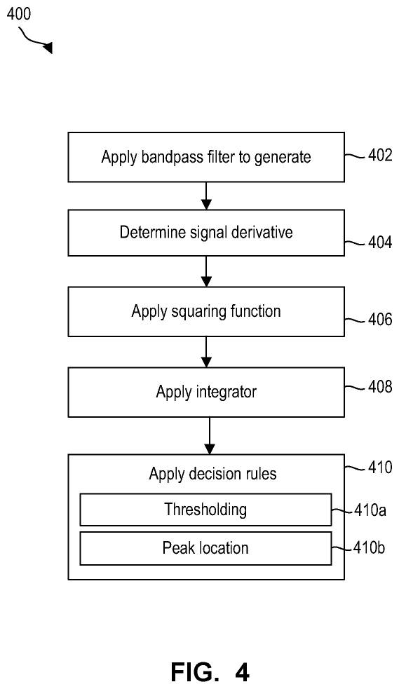

At 306, an estimation model may be applied to the pre-filtered and digitized ECG signal to determine one or more heartbeat parameters. While any suitable heartrate monitoring model can be used to estimate heartbeat parameters—in at least one embodiment, a low-power “tiny” AI model is applied, as discussed in greater detail herein with reference to FIG. 4. In various cases, the model may take, as inputs, the SQI to determine which portions of the ECG signal can be used to generate high quality estimates.

At 308, the determined heartbeat parameters may be output. For example, outputting the heartbeat parameters can comprise displaying the heartbeat parameters, in real-time or near real-time, on a monitoring device display 208a. In other cases, the monitoring device 104 may be configured to generate an alert or notification (i.e., visual or auditory) if the heartbeat parameters are in a normal or abnormal range. In still other cases, the output heartbeat parameters may be transmitted to an external computer terminal 112a and/or server 112b. For example, the heartbeat parameters may be stored on a memory 204b of computer terminal 112a and/or server 112b for later retrieval or processing. In some cases, a display of the computer terminal 112a may include a GUI for displaying the data to a user of the terminal 112a for further analysis and observation.

At 310, in some cases, intervention can be applied to the monitored pediatric subject based on the output at act 308. For instance, if the output indicates an abnormal heartbeat range, the intervention can include applying respiratory to the pediatric subject, or otherwise applying resuscitation.

In some embodiments, the processing by device processor 202a, in executing method 300, may consume an average from 1 mA to a few 100 mAh, based on the application. In turn, the method 300 may be considered to be a low-power method that is adapted for the low processing capabilities of the device processor 202a as well as the low energy storage of the energy storage unit 214a.

Reference is now made to FIG. 4, which shows an example embodiment of a process flow for a method 400 for analyzing ECG data to determine one or more heartbeat parameters. Method 400 may be characterized as a modified version of the Pan-Tompkins technique for analyzing heartrate. Method 400 may be performed by the processor 202a of the monitoring device 104, and may be implemented at act 306 of FIG. 3.

At 402, a bandpass filter is applied to the received ECG signal. As explained previously, the received ECG signal may correspond to the pre-filtered and digitized signal received at act 302 in FIG. 3 In some cases, the bandpass filter may have a bandpass range of 5 to 15 Hz or what is suitable for pediatric patients.

At 404, a derivative of the ECG signals is determined to generate a derived ECG signal, which is used to emphasize signal features.

At 406, a squaring function is applied to the derived ECG signal to generate a squared ECG signal. The squaring function can emphasize or pronounce the ECG signal peaks, which can assist in R-peak detection.

At 408, an integrator function is applied to the squared ECG signal to generate an integrated ECG signal. Applying the integrator function can act as a smoothing operator to minimize noise residue retained throughout the signal analysis process.

At 410, one or more decision rules are applied to the integrated ECG signal to determine if the heartrate is abnormal based on one or more determined heartbeat monitoring parameters. In at least one embodiment, act 406 may involve initially thresholding 410a the integrated ECG signal to reduce the signal to a plurality of signal peak, and further identifying (or counting) the number of signal peaks 410b (i.e., R-peak detection) to determine properties of the ECG signal. In various cases, an adaptive thresholding technique is used such that whenever ECG signals are collected, the threshold changes based on changes in noise. The threshold may also be adjusted based on subject-specific factors and considerations. In some embodiments, act 410 may include determining parameters such as peak locations, heartrate, heartrate variability (HRV) and RR distance between consecutive R-peaks. The parameters can then be further analyzed by a lower-power machine learning model (i.e., a tiny AI model) to determine if the pediatric subject is in critical condition. In some cases, a linear discriminant analysis is used where features are derived from the ECG signal, and classifier coefficients are estimated by a novel optimization technique.

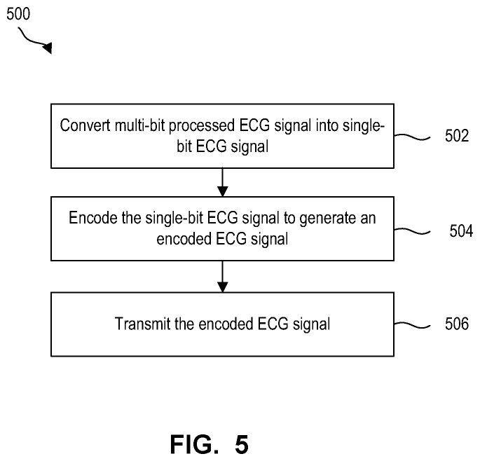

Reference is now made to FIG. 5, which show a process flow for an example embodiment for a method 500 for signal compression. Method 500 may be performed by the processor 202a of the monitoring device 104, i.e., in the process of executing the signal analysis software 216a. In particular, method 500 allows for low data bit transmission at act 308 of method 300 of FIG. 3. The compression can reduce the processed heartbeat signal such that only signal peak data is transmitted, rather than the complete heartbeat signal.

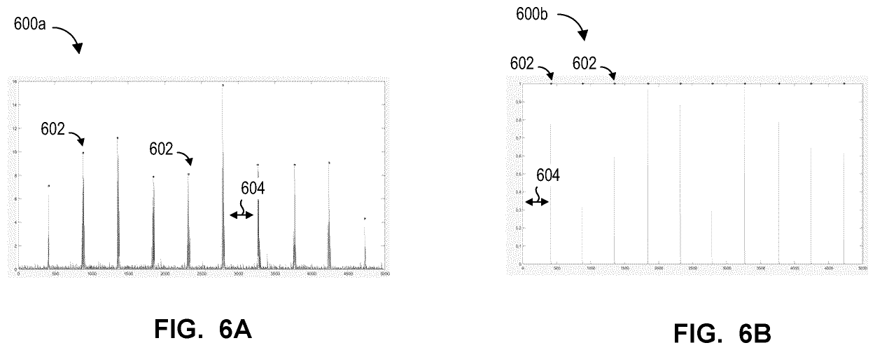

At 502, the multi-bit processed ECG signal (i.e., the ECG signal generated at act 308) is converted into a single-bit ECG signal. For instance, as explained previously, the ECG signal may be a 12-bit signal (plot 600a in FIG. 6A), and accordingly, the 12-bit signal may be converted into a 1-bit signal (plot 600b in FIG. 6B).

As shown in plot 600b, the 1-bit signal may only emphasize the signal peaks, such that the signal peaks 602b are expressed by a binary bit “1”, and all other regions of the signal 604 are represented by a binary bit of “0”. In some cases, this mapping is assisted by the thresholding performed at act 410a, such that values above the threshold are mapped to a binary bit “1” and values below the threshold are mapped to a binary bit of “0”. To this end, it has been appreciated that retaining only signal peaks data may be sufficient to determine necessary heartbeat parameters, including heartrate and heartrate variability based on the location and frequency of the peaks.

Table 1, below, demonstrates the resulting compression ratio and compression percentage for different time lengths of ECG signal acquisition (i.e., 5 minute versus 30 seconds). As shown, the compression at 502 can significantly reduce the size of the signal.

TABLE 1
Compression Values

| | 5 min signal | 30 second signal |
|---|---|---|
| Compression Ratio | 160.9936 | 41.179666 |
| Compression % of original | 0.6211% | 1.4746% |
| Signal | | |

At 504, the single-bit ECG signal may be further encoded to enable transmission. For example, the single-bit ECG signal may be encoded using a Lempel-Ziv encoding algorithm. In this manner, the encoded signal may be decoded at the other end using a complementary decoding algorithm. In various cases, a Lempel-Ziv encoding algorithm is used as it performs well on runs of binary bits. As well, the Lempel-Ziv encoding algorithm provides additional benefits including: (i) being a hardware friendly encoding algorithm, and can be implemented on a low power processor; and (iii) ability to retain all signal information, accordingly no information is lost in the encoding process.

At 506, the encoded ECG signal is transmitted, i.e., to a remote computer terminal 112a and/or server 112b.

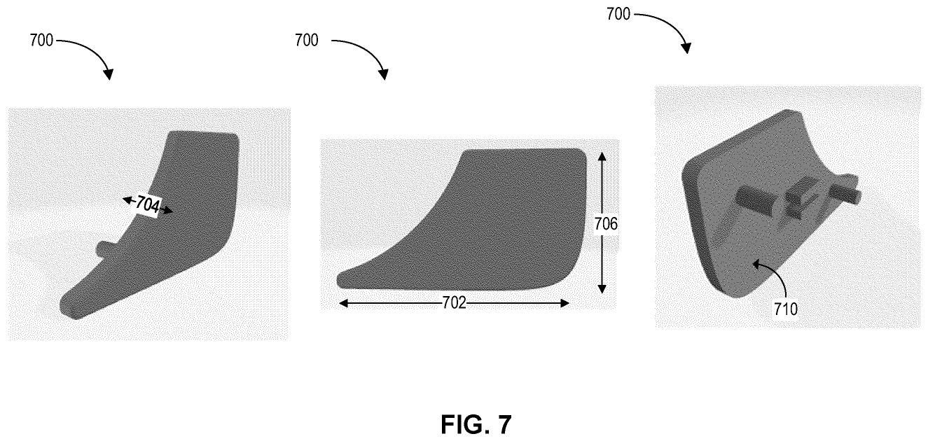

Reference is now made to FIG. 7, which shows a schematic illustration of an example embodiment of a 3D printed dry electrode 700. The dry electrode is an example of an electrode 220a located in the heartbeat monitoring device 104, as shown in FIG. 2.

As explained previously, it has been appreciated that dry electrodes may achieve better comfortability for users (i.e., as compared to wet electrodes), and are generally better suited for neonatal applications. However, there are certain challenges in manufacturing low cost dry electrodes which are suited for mass production, and which can produce high quality ECG signals to maintain a high standard of clinical relevance for medical diagnosis. To overcome these challenges, the inventors have realized a unique, low-cost electrode that can be 3D printed using common 3D printing material and 3D printing appliances.

In at least one embodiment, the dry electrode 700 may be manufactured from a 3D printed filament with electrically conductive properties. In some cases, the conductive polylactic acid (PLA) is a conductive carbon polymer that is semi-flexible film. Conductive PLA firm is a widely and commonly available film type.

In at least one embodiment, the dry electrode 700 is 3D printed using a 3D printer such as an ANYCUBIC™ i3 S 3D printer. The printer may have a nozzle temperature of about 215° C., a heated bed temperature of about 60° C., a print speed of about 25 mm/s and a fill ratio of 100%. Using these operational printed parameters, the electrodes can be manufactured in less than about 10 minutes, and with minimal resources. After 3D printing, the electrodes are otherwise ready for use, and may not require any other structural processing. This, in turn, facilitates rapid manufacturing of dry electrodes for use in conjunction with the monitoring device 104.

In some embodiments, the dry electrodes 700 can have length 702×width 704×height 706 dimensions of 32 millimeters×6 millimeters×18 millimeters, respectively, which provides large surface area for acquiring ECG signals. Further, the dry electrodes can have an electrical volumetric resistivity across the surface contact front area 710 in a range of about 1,000 to 1,400 Ohms. This allows matching the variable resistance of the subject's body with the electrode in order to acquire a high quality signal from the body. In at least some embodiments, there may be hardware components placed within the electrode 700 to help provide this match. For example, the electrode 700 may include various impedance matching electronics (i.e., resistors and op-amps) that are controlled by the processor 202a, of monitoring device 104—either automatically, or based on inputs received from the device input interface 212a.

In some embodiments, the electrode 700 can be placed in a shield, such as a Faraday cage, to reduce noise infiltrating the acquired ECG signal.

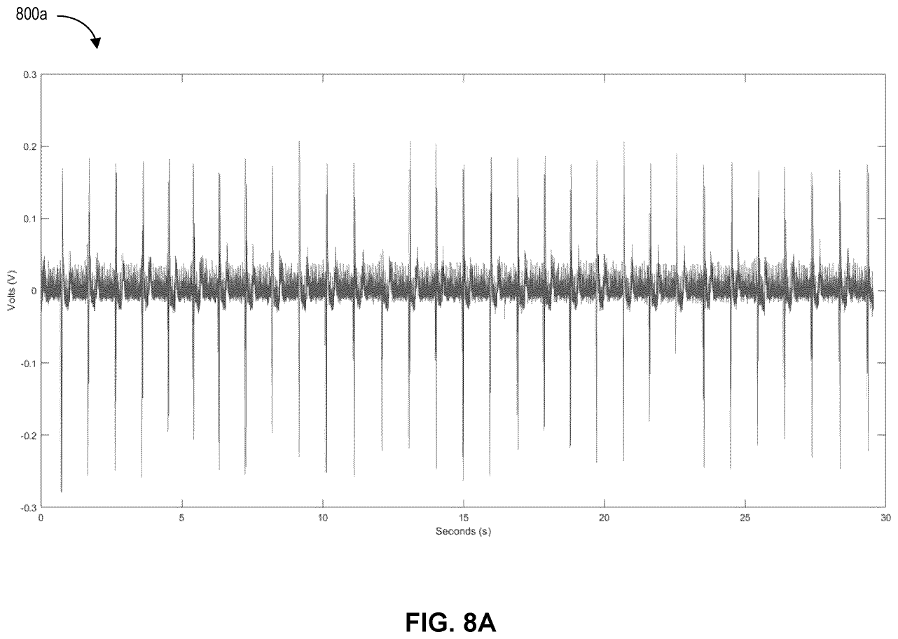

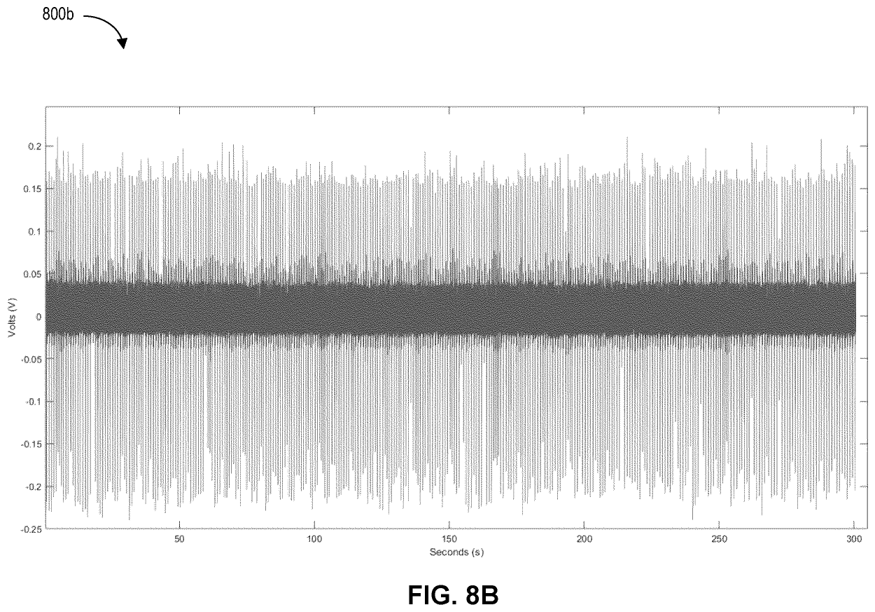

Reference is now made to FIGS. 8-10 which show various plots that illustrate the feasibility of using a 3D printed electrode 700 in heartrate detection for various applications, including consumer and medical applications.

FIGS. 8A and 8B show example plots 800a, 800b, respectively, of ECG signals acquired from a single 3D printed dry electrode 700 applied to a pediatric subject, and showing recorded voltage versus time. Plots 800a, 800b may be acquired using a signal acquisition device similar to the monitoring device 104. Plot 800a shows a thirty second (30 second) acquisition window, while plot 800b shows a five minute acquisition window. The ECG signals in plots 800a, 800b are illustrated after pre-filtering by filtering hardware 206a in FIG. 2.

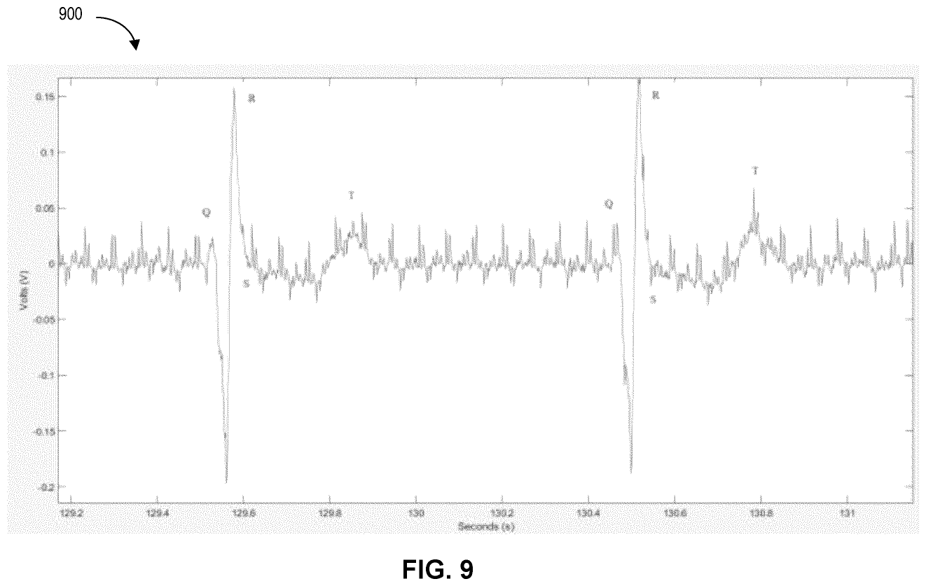

FIG. 9 shows an example plot 900 of a snapshot of two ECG cycles from the plot 800b, and labelled with the QRS complex and T-wave. As shown, the QRS complex and T-waves are easily visible and identifiable from the ECG signal generated by the 3D dry electrode 700.

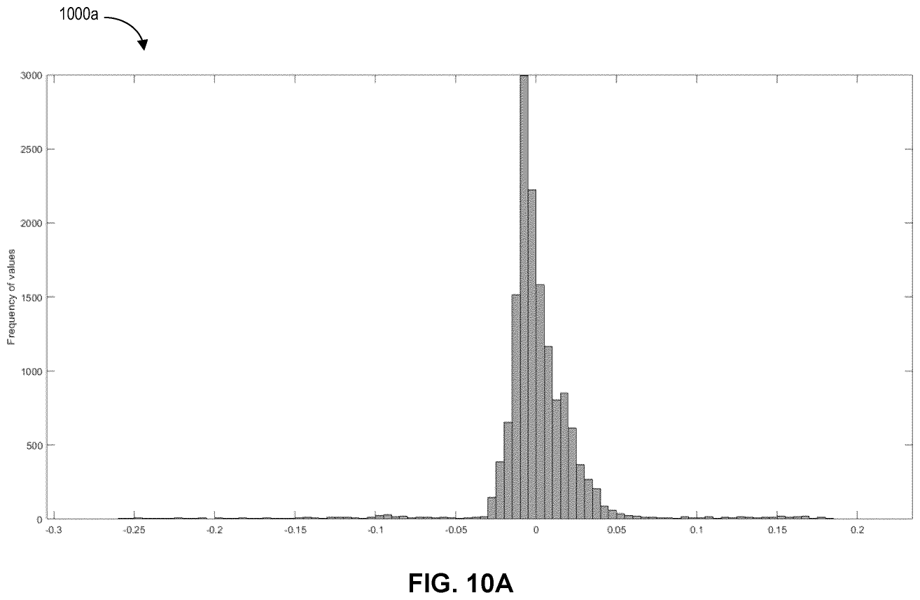

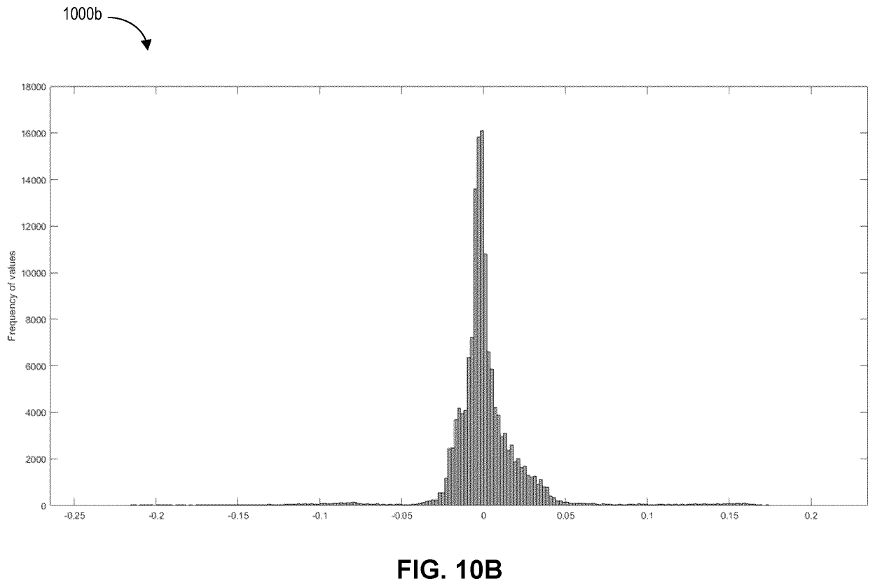

FIGS. 10A and 10B show example histogram plots 1000a, 1000b, respectively, generated from the plots 800a, 800b of FIG. 8, respectively. The histograms 1000a, 1000b represent the sSQI of the acquired signals. As shown, the thirty second and five minute acquisition windows—represented by histograms 1000a, 1000b—are slightly skewed to the right. The thirty second recording shows an sSQI of −1.24, representing heavy skewness while the five minute recording shows a skewness of −0.62 representing moderate skewness.

As shown in Table 2, below, all SQIs improve with a longer duration acquisition window. For example, kSQI and sSQI decrease with increased recording length. Both values are related to the presence of randomness in a single. Normal ECG signals are periodic in nature, which means that with longer signal length, the presence of a repeating rhythm is more evident than the noise/randomness present. In respect of SD, activity, mobility—the values are relatively stable across all signals. These occur because the morphology of the ECG does not change over time, which indicates that the person's ECG is normal. Importantly, the SD, activity and mobility signal parameters are used in feature selection algorithms to determine abnormalities in the ECG. To this end, the five minute signal shows the highest signal-to-noise (SNR) ratio (i.e., 10.05 dB). The heartrate was determined using the methods 300, 400, and as shown, there is an increase in estimated heartrate with increased recording time where thirty seconds, one minute and five-minute signals showed 63, 65 and 66 beats per minute (BPM).

TABLE 2
SQI and Detected Heartrate for Various Acquisition
Time Windows Using a 3D Printed Dry Electrode

| | 30 Seconds | 1 Minute | 5 Minutes |
|---|---|---|---|
| kSQI | 21.56 | 19.45 | 18.90 |
| sSQI | −1.24 | −0.75 | −0.62 |
| SD | 0.0344 | 0.0335 | 0.0317 |
| Activity | 0.0012 | 0.0011 | 0.0010 |
| Mobility | 3.18 | 3.26 | 3.15 |
| Standard of | 2.83 × 10−4 | 1.94 × 10−4 | 8.18 × 01−5 |
| Deviation Error | | | |
| SNR in dB | 12.33 | 12.77 | 13.05 |
| HR in bpm | 63 | 65 | 66 |

In view of the foregoing, the inventors have appreciated the applicability of 3D printed dry electrodes (e.g., dry electrode 700 in FIG. 7) in ECG acquisition, and for the purposes of monitoring heartbeat parameters in subjects, including pediatric subjects. The 3D dry electrodes provide advantages over conventional dry electrodes, or wet electrodes. For example, as previously stated—as contrasted to conventional dry electrodes, the 3D dry electrodes are easier, cheaper and faster to manufacture using widely available 3D printing tools. In turn, the 3D printed electrodes are easily manufactured and deployed. Further, as contrasted to wet electrodes, the 3D printed electrodes can be applied to a subject's skin with no previous skin preparation, which finds particular significance for sensitive skin for pediatric subjects.

While the above description describes features of example embodiments, it will be appreciated that some features and/or functions of the described embodiments are susceptible to modification without departing from the spirit and principles of operation of the described embodiments. For example, the various characteristics which are described by means of the represented embodiments or examples may be selectively combined with each other. Accordingly, what has been described above is intended to be illustrative of the claimed concept and non-limiting. It will be understood by persons skilled in the art that other variants and modifications may be made without departing from the scope of the invention as defined in the claims appended hereto. The scope of the claims should not be limited by the preferred embodiments and examples, but should be given the broadest interpretation consistent with the description as a whole.

1. A system for pediatric heartbeat monitoring, comprising:
at least one electrode configured to be applied to a pediatric subject;
at least one processor coupled to the at least one electrode, the at least one processor configured for:
receiving, from the at least one electrode, an input electrocardiogram (ECG) signal;
determining a signal quality index (SQI) associated with input ECG signal;
applying a bandpass filter to the input ECG signal to generate a filtered ECG signal;
determining a derivative of the filtered ECG signal to generate a derived ECG signal;
applying a squaring function to the derived ECG signal to generate a squared ECG signal;
applying an integrator to the derived ECG signal to generate an integrated ECG signal; and
applying one or more decision rules to the integrated ECG signal to output one or more heartbeat parameters associated with the subject.

2. The system of claim 1, wherein the at least one processor is configured to output the one or more heartbeat parameters in real-time, or near real-time.

3. The system of claim 1, further comprising a hardware circuit filter coupled between the at least one electrode and the at least one processor, wherein the hardware circuit filter receives an ECG signal from the at least one electrode and generates a pre-filtered ECG signal, and the input ECG signal comprises the pre-filtered ECG signal.

4. The system of claim 3, wherein the hardware circuit filter is a bandpass filter.

5. The system of claim 4, wherein the bandpass filter has a passband range of 3 to 48 Hz.

6. The system of claim 1, wherein the bandpass filter has a passband range of 5 to 15 Hz.

7. The system of claim 1, wherein the one or more output heartbeat parameters comprise one or more of heartrate, heartrate variability, RR interval and R-peak locations, and an indication of abnormal heartrate.

8. The system of claim 1, wherein the SQI is one of kurtosis SQI (kSQI), skewness SQI (sSQI), a histogram, an activity measure, a mobility measure, a signal to noise ratio (SNR), a LZW complexity measure and a fractal dimension measure.

9. The system of claim 1, wherein the input ECG signal is a 12-bit ECG signal.

10. The system of claim 1, wherein the at least one processor is further configured to apply thresholding to the integrated ECG signal and to further convert the signal into a one-bit signal.

11. The system of claim 1, wherein the at least one processor is further configured to:
encode the one or more output heartbeat parameters using a varied Lempel-Ziv encoding algorithm to generate encoded output parameters; and
transmit the one or more encoded output parameters to an external device.

12. The system of claim 1, wherein the at least one electrode comprises a 3D printed dry electrodes printed from conductive polylactic acid (PLA) film.

13. The system of claim 12, wherein each of the at least one 3D printed dry electrodes has length, height and width dimensions of 32 millimeters, 18 millimeters and 6 millimeters, respectively.

14. The system of claim 1, wherein the dry electrodes are manufactured using a nozzle temperature of about 215° C., a heated bed temperature of about 60° C., a print speed of about 25 mm/s and a fill ratio of 100%.

15. A method for pediatric heartbeat monitoring comprising:
receiving, from the at least one electrode, an input electrocardiogram (ECG) signal;
determining a signal quality index (SQI) associated with input ECG signal;
applying a bandpass filter to the input ECG signal to generate a filtered ECG signal;
determining a derivative of the filtered ECG signal to generate a derived ECG signal;
applying a squaring function to the derived ECG signal to generate a squared ECG signal;
applying an integrator to the derived ECG signal to generate an integrated ECG signal; and
applying one or more decision rules to the integrated ECG signal to output one or more heartbeat parameters associated with the subject.

16. The method of claim 15, wherein the one or more heartbeat parameters are output in real-time, or near real-time.

17. The method of claim 15, further comprising:
receiving an ECG signal from the at least one electrode and
generating, via a hardware circuit filter, a pre-filtered ECG signal, and the input ECG signal comprises the pre-filtered ECG signal.

18. The method of claim 17, wherein the hardware circuit filter is a bandpass filter.

19. The method of claim 18, wherein the bandpass filter has a passband range of 3 to 48 Hz.

20. The method of claim 15, wherein the bandpass filter has a passband range of 5 to 15 Hz.

21. (canceled)

22. (canceled)

23. (canceled)

24. (canceled)

25. (canceled)

26. (canceled)

27. (canceled)

28. (canceled)
